(19) United States
(12) Patent Application Publication
(10) Pub. No.: US 2014/0200492 A1
(43) Pub. Date: Jul. 17, 2014

(54) FOOT ORTHOSIS

(71) Applicant: Midwest Orthotic Services, LLC., South Bend, IN (US)

(72) Inventors: Joshua Paul Ahlstrom, Noblesville, IN (US); Scott Robert Hover, Manitou Beach, MI (US)

(73) Assignee: Midwest Orthotic Services, LLC., South Bend, IN (US)

(21) Appl. No.: 14/157,806

(22) Filed: Jan. 17, 2014

Related U.S. Application Data
(60) Provisional application No. 61/753,621, filed on Jan. 17, 2013.

(51) Int. Cl. A61H 23/02; A61F 5/01

(52) U.S. Cl. CPC A61H 23/02; A61F 5/0127
USPC 601/46

(57) ABSTRACT

A foot orthosis includes at least one pressure sensing switch, at least one vibrating member, a controller and a power source. The pressure sensing switch is activated as the wearer walks and applies force to the switch. The switch activates the vibrating member, which sends a vibrating pulse to stimulate the patient's foot. The controller can be programmable and controls the frequency and duration of the vibration.

The present invention generally relates to orthotic devices and, in particular, to a foot orthosis.

### BACKGROUND AND SUMMARY OF THE INVENTION

A variety of diseases can lead to neuropathies and/or loss of sensation of the feet and lower extremities in certain patients. This loss of sensory function can greatly diminish or destroy the patient's ability for proprioception, and consequently, makes walking and postural stability much more difficult for them and increases their risk of falling and other injuries.

In one embodiment of the present invention, a vibrating foot orthosis is used to improve or restore sensory perception in the feet, which in turn improves or restores proprioception. The orthosis includes an insole for insertion into an item of footwear worn by the patient. The orthosis can include at least one pressure sensing switch, at least one vibrating member, a controller and a power source. The pressure sensing switch is activated when the sole of the patient's footwear contacts the ground. The switch in turn activates the vibrating member, which sends a brief, vibrating pulse to stimulate the patient's foot.

In one embodiment of the invention, the orthosis produces the desired level of vibration throughout the entire surface of the patient's sole. In other embodiments, the orthosis targets the vibration to limited areas of the patient's foot. In certain embodiments of the invention, the orthosis is designed to be in constant contact with substantially the entire sole of the patient's foot. In other embodiments, the orthosis only contacts a portion, or selected portions, of the patient's foot. The orthosis can be a single, unitary device. Alternatively, the orthosis can include a plurality of individual sections positioned at the desired locations in the patient's footwear.

In one embodiment of the invention, a foot orthosis includes first, second, third, fourth and fifth layers constructed from a variety of materials. Each layer has a first surface and a second surface. The first surface of the second layer is in contact with the second surface of the first layer. The first surface of the third layer is in contact with the second surface of the second layer. The first surface of the fourth layer is in contact with the second surface of the third layer. The first surface of the fifth layer is in contact with the second surface of the fourth layer. The orthosis further includes a power source located in one of the layers, a vibrating device located in one of the layers and a force-sensitive switch located in one of the layers for activating the vibrating device upon activation of the switch. The orthosis also includes a programmable controller for controlling the frequency and duration of vibration of the vibrating device upon activation of the switch.

In certain embodiments, the first layer may be constructed from polyethylene foam, the second layer may be constructed from polypropylene, the third layer may be constructed from medium density polyethylene foam, the third layer may be constructed from high density polyethylene foam and the fifth layer may be constructed from fabric.

In other embodiments of the invention, a foot orthosis includes a heel end, a toe end and an arch section having a first end and a second end located between the heel end and the toe end. The fourth layer extends from a point adjacent the heel end to a point between the toe end and the second end of the arch section. The fourth layer may be more rigid than the first layer.

In another embodiment, a foot orthosis includes a heel end, a toe end and an arch section located between the heel end and the toe end. The third layer is substantially coextensive with the length of the arch section.

In other embodiments, a foot orthosis includes a heel end, a toe end and an arch section having a first end and a second end located between the heel end and the toe end. The fifth layer extends from a point between the heel end and the first end of the arch section to a point adjacent the toe end. The fourth layer may be more rigid than the fifth layer.

In certain embodiments, the vibrating device and the switch are located in the same layer. In other embodiments, the vibrating device is located in a layer above the layer in which the switch is located. In some embodiments, the vibrating device is located in a layer below the layer in which the switch is located. In other embodiments, the controller is located between the fourth and fifth layers.

In certain embodiments, the vibrating device is a vibrating motor.

In other embodiments, the orthosis is configured to fit within an article of footwear and is removable therefrom. In other embodiments, the orthosis is integral with an article of footwear.

In some embodiments, the controller is located in one of the layers. In certain embodiments, the controller communicates with the vibrating device via a wireless connection.

In one embodiment of the invention, a foot orthosis includes a power source, a vibrating device, a switch for activating the vibrating device and a programmable controller for controlling the frequency and duration of vibration of the vibrating device upon activation of the switch.

In certain embodiments of the invention, the switch is a force-sensitive switch.

In some embodiments of the invention, the orthosis further includes an article of footwear. In some embodiments, the vibrating device and switch are integral with the article of footwear. In other embodiments, the power source is integral with the article of footwear. In other embodiments, the controller is integral with the article of footwear.

In some embodiments, the orthosis has an upper surface and a lower surface, the switch is located on the upper surface and the vibrating device is located on the lower surface.

In one embodiment of the invention, a foot orthosis includes means for producing vibration, means for activating the means for producing vibration, means for controlling the frequency and duration of the vibration produced by the means for producing vibration and means for securing the means for vibration adjacent a human foot.

It will likely be desirable for the patient to wear the device regularly over an extended period of time. Thus, the orthosis is preferably constructed from durable materials. In one embodiment, at least portions of the orthosis are water proof and sweat proof to protect the switches, vibrating members, controller and power source from damage caused by moisture.

Other features of the present invention will be apparent from the following description and accompanying drawings.

### BRIEF DESCRIPTION OF THE DRAWINGS

FIG. 1 is a side elevational view of a foot orthosis according to one embodiment of the present invention.

FIG. 2 is a top plan view of the foot orthosis shown in FIG. 1.

FIG. 3 is a cross-sectional view taken along the length of the orthosis shown in FIG. 1.

FIG. 4 is a circuit diagram for a controller that is a component of a foot orthosis according to one embodiment of the present invention.

FIG. 5 is a circuit board lay out of the circuit shown in FIG. 4.

FIGS. 6A-6D illustrate another embodiment of a foot orthosis according to the present invention.

### DETAILED DESCRIPTION OF EMBODIMENTS OF THE INVENTION

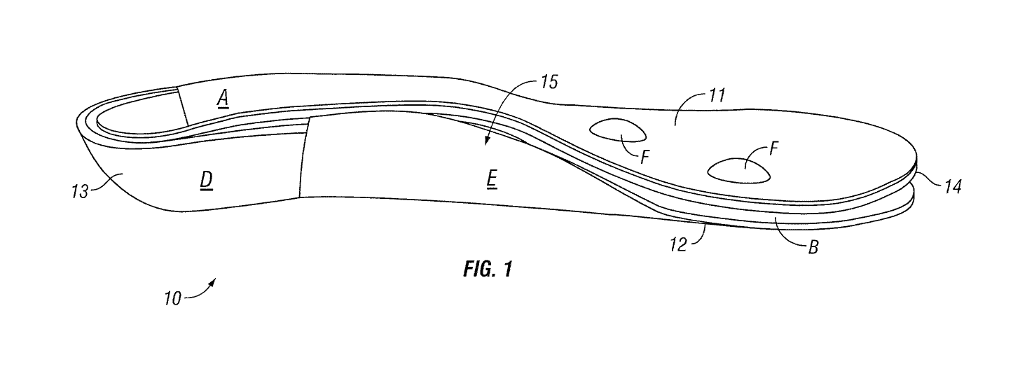

Turning to FIGS. 1-3, a foot orthosis 10 according to one embodiment of the present invention includes a first or upper surface 11 for contacting the user's foot or a sock, stocking or similar covering worn over the foot, a second or lower surface 12 for contacting the interior of the patient's shoe, boot or other footwear, a first or heel end 13, a second or toe end 14 and an arch section 15.

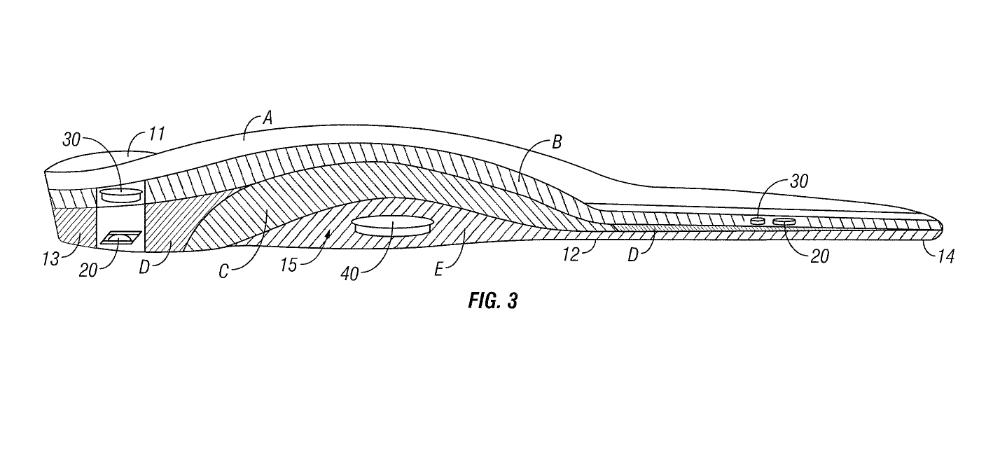

In the embodiment shown, orthosis 10 is substantially constructed from five different materials. The uppermost portion of the orthosis 10, which includes first or upper surface 11, is, in the embodiment shown, constructed from a polyethylene foam material indicated by reference letter A. (FIG. 3) A second or intermediate layer 16 is constructed from a polypropylene material indicated by reference letter B. A third layer substantially coextensive with arch section 15 is constructed from medium density polyethylene foam indicated by reference letter C. A fourth layer that extends from heel end 13 toward toe end 14 just beyond arch section 15 is constructed from high density polyethylene foam indicated by reference letter D. A fifth layer extending from just beyond the end of arch section 15 nearest heel end 13 to toe end 14 is constructed from a durable fabric material indicated by reference letter E.

It should be noted that these materials were selected so as to provide the desired levels of comfort, support and durability. For example, heel end 13 of orthosis 10 will typically be subjected to greater forces during walking than other areas of orthosis 10. Arch section 15 must provide sufficient support. Thus, it is desirable to use a realatively strong, less flexible material such as high density polyethylene foam to provide durability and support in these areas of orthosis 10. However, the relatively rigid nature of this material would make it difficult for the patient's toes to flex if it extend all the way to toe end 14 of orthosis 10. Thus, this material layer terminates just beyond arch section 15. Similarly, a relatively soft, flexible polyethylene foam is used for the first layer in this embodiment so as to provide greater comfort to the patient. Any of a number of materials and combinations of material can be used for orthosis 10 that provide the desired levels of comfort, durability and performance.

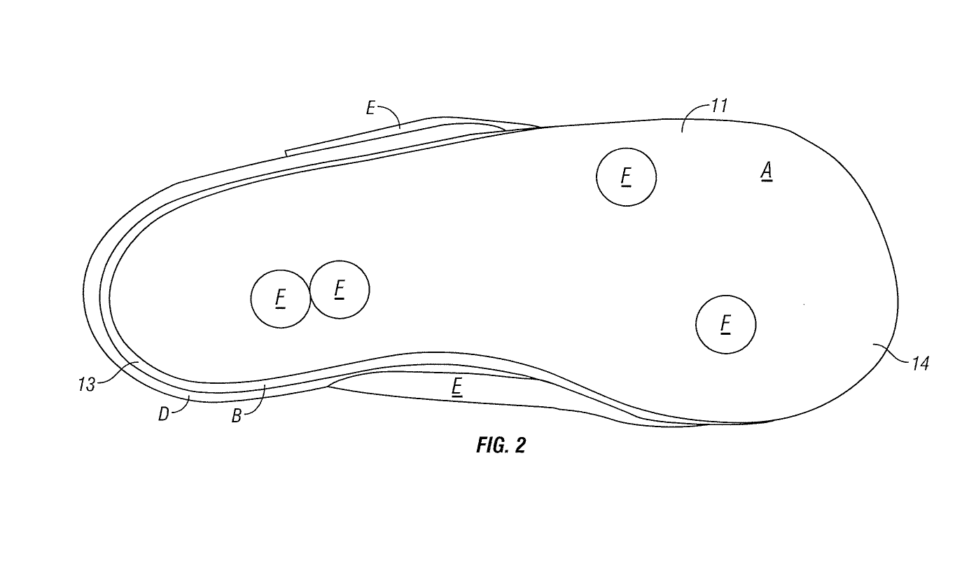

In the embodiment shown, a plurality of force-sensitive switches 20 are embedded in orthosis 10. Switches 20 should be embedded sufficiently close to lower surface 12 to ensure that they are consistently activated as the patient walks. The placement of switches 20 will vary based on the type of materials used for orthosis 10. A plurality of vibrating devices 30 are positioned at the locations at which it is desired to deliver a vibrating pulse for the particular patient's treatment. Vibrating devices 30 should be embedded sufficiently close to upper surface 11 of orthosis 10 so as to ensure the desired level of stimulation for the particular patient's program of treatment. Reference letter F in FIG. 2 illustrates the placement of vibrating devices 30 for the embodiment shown. In this embodiment, two vibrating devices 30 are positioned on the interior and exterior ends of the ball of the foot and two vibrating devices 30 are positioned adjacent each other along the midline of the foot, slightly in front of the heel. Other locations and additional vibrating devices may be utilized depending upon the particular patient's program of treatment. In one embodiment of the invention, vibrating devices 30 are vibrating motors. In one embodiment of the invention, wires or other electrical conductors (not shown) connect switches 20 with vibrating devices 30 so as to activate the vibrating devices upon activation of the switches.

As shown in FIG. 3, a controller 40 is positioned beneath arch section 15 and is secured in place and covered by the fabric layer. Controller 40 is preferably programmable so that the duration and strength/frequency of the vibrations delivered by vibrating devices 30 can be tuned to the program of treatment for individual patients. Controller 40 is preferably integrated with a power source, such as a lithium battery 50.

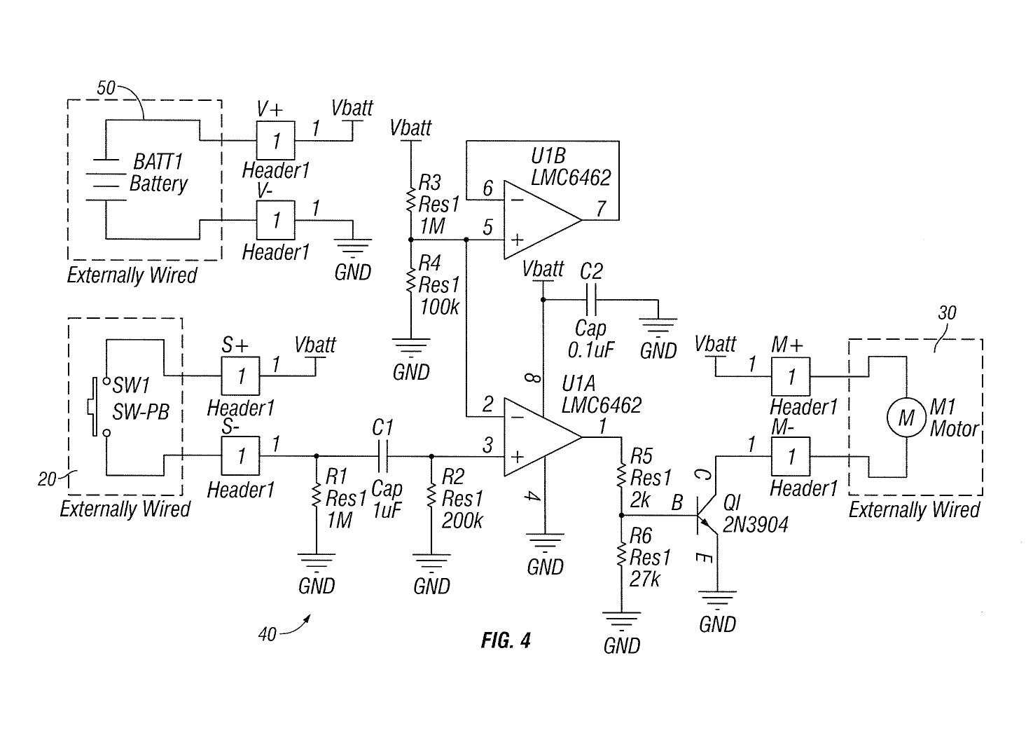

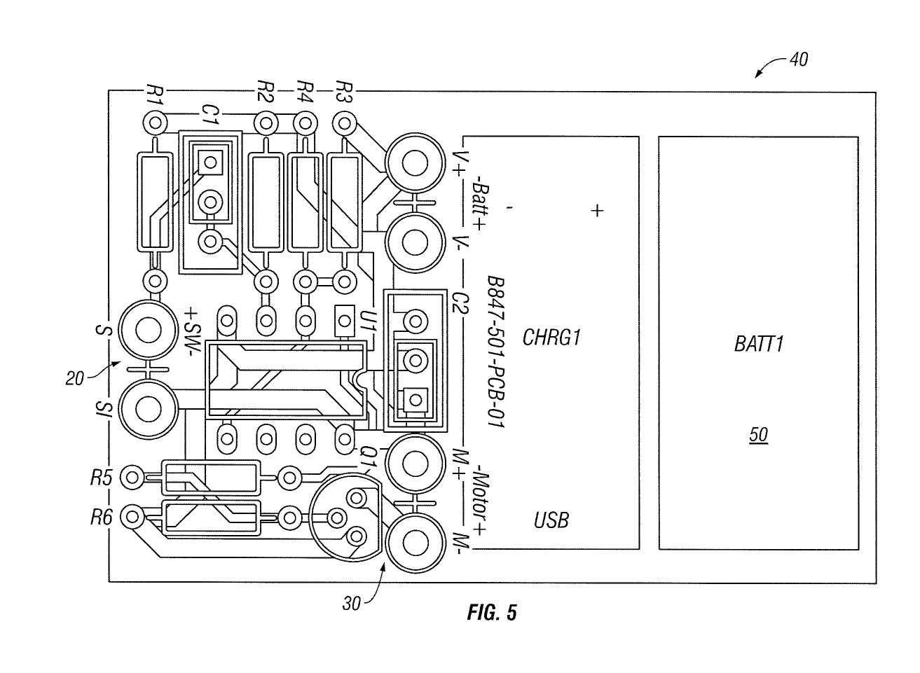

FIGS. 4 and 5 show, respectively, a circuit diagram and circuit board layout for a controller according to one embodiment of the present invention. It should be noted that other circuits and layouts can be utilized that provide the desired functionality for orthosis 10.

In use, foot orthosis 10 is inserted into the patient's footwear. Each time the outer sole of the patient's footwear makes contact with the ground and sufficient body weight is placed on upper surface 11 to activate pressure sensitive switches 20 the switches 20 in turn cause activation of vibrating devices 30. This causes vibrating devices 30 to send a brief vibrating pulse through orthosis 10 to stimulate the patient's foot. Depending on the program of treatment, the vibrating pulse will typically last about 2 seconds.

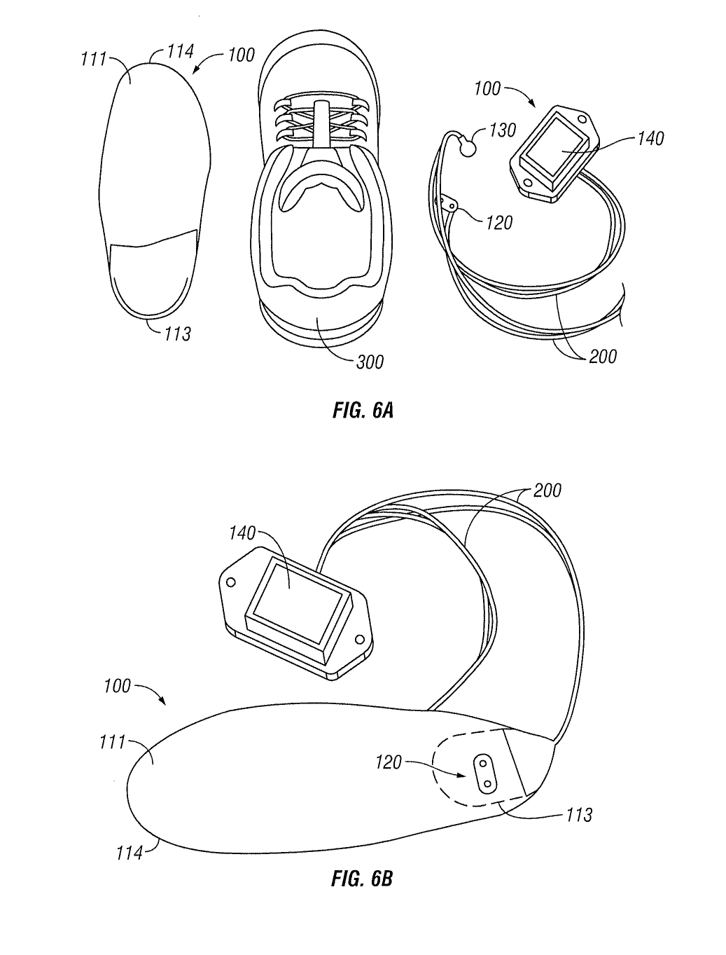

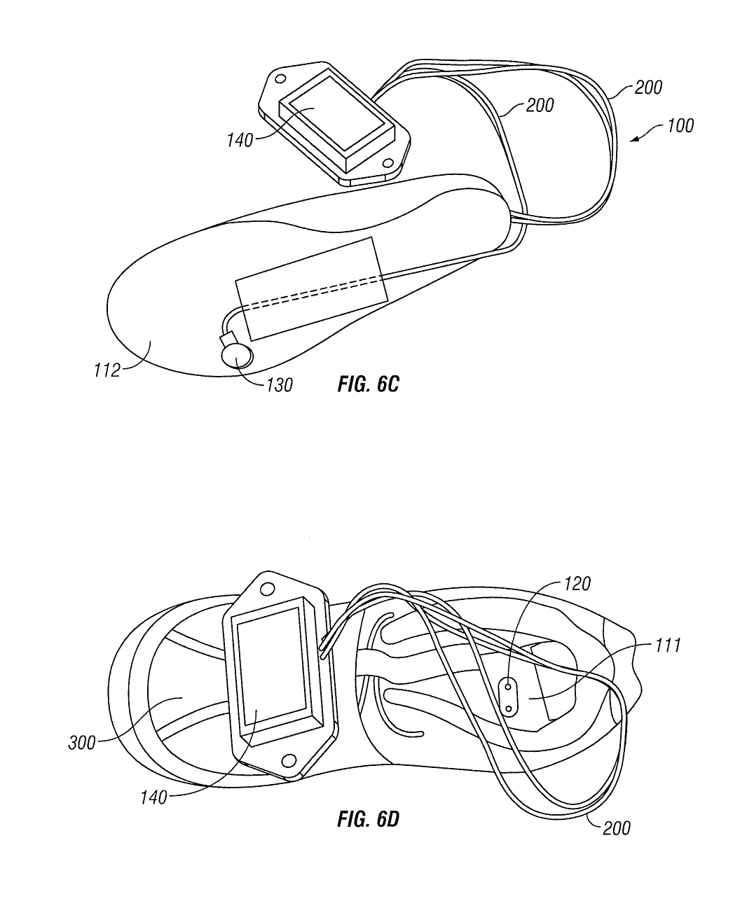

FIGS. 6A-6D show an alternative embodiment of a foot orthosis according to the present invention. In this embodiment, orthosis 100 includes a first or upper surface 111, a second or lower surface 112, a switch 120, a vibrating device 130 and a controller 140. Switch 120 and vibrating device 130 are connected to controller 140 via wires 200. A power source (not shown) may be incorporated into the housing of controller 140. Switch 120 is secured on first surface 111. In the embodiment shown, switch 120 is placed adjacent heel end 113. Vibrating device 130 is secured to second surface 112. Note that a covering or padding may be placed over switch 120 and vibrating device 130 for increased comfort and to protect those devices. Orthosis 110 may be inserted in an article of footwear 300 as shown in FIG. 6D and controller 140 may be secured to the article of footwear. When the wearer walks, pressure on switch 120 will activate it, which will in turn cause activation of vibration device 130 as described above.

Although the present invention has been shown and described in detail the same is to be taken by way of example only and not by way of limitation. Numerous changes can be made to the embodiments shown without departing from the scope of the invention. For example, cloth layer E can be eliminated and controller 40 can be embedded in one of the remaining layers of orthosis 10. Alternatively, controller 40 could simply be adhered to orthosis 10 at a desired location. If wireless technology is used, controller 10 can be located elsewhere on the patient or carried in the patient's pocket instead of being connected to orthosis 10. The switches, controller and vibrating devices could also be integrated into an item of footwear instead of being produced as a separate insert. The present invention may be further modified within the spirit and scope of this disclosure. The application is, therefore, intended to cover any variations, uses, or adaptations of the invention using its general principles. Further, this application is intended to cover such departures from the present disclosure as come within known or customary practice in the art to which this invention pertains.

1. A foot orthosis, including:
a first layer constructed from a first material, the first layer having a first surface and a second surface;
a second layer constructed form a second material, the second layer having a first surface in contact with the second surface of the first layer and a second surface;
a third layer constructed from a third material, the third layer having a first surface in contact with the second surface of the second layer and a second surface;
a fourth layer constructed from a fourth material, the fourth layer having a first surface in contact with the second surface of the third layer and a second surface;
a fifth layer constructed from a fifth material, the fifth layer having a first surface in contact with the second surface of the fourth layer and a second surface;
a power source located in one of the layers;
a vibrating device located in one of the layers;
a force-sensitive switch located in one of the layers for activating the vibrating device upon activation of the switch; and
a programmable controller for controlling the frequency and duration of vibration of the vibrating device upon activation of the switch.

2. The foot orthosis according to claim 1, wherein the first material is polyethylene foam.

3. The foot orthosis according to claim 2, wherein the second material is polypropylene.

4. The foot orthosis according to claim 3, wherein the third material is medium density polyethylene foam.

5. The foot orthosis according to claim 4, wherein the fourth material is high density polyethylene foam.

6. The foot orthosis according to claim 5, wherein the fifth material is fabric.

7. The foot orthosis according to claim 1, wherein the orthosis includes a heel end, a toe end, an arch section having a first end and a second end located between the heel end and the toe end and wherein the fourth layer extends from a point adjacent the heel end to a point between the toe end and the second end of the arch section.

8. The foot orthosis according to claim 7, wherein the fourth material is more rigid than the first material.

9. The foot orthosis according to claim 1, wherein the orthosis includes a heel end, a toe end, an arch section located between the heel end and the toe end and wherein the third layer is substantially coextensive with the length of the arch section.

10. The foot orthosis according to claim 1, wherein the orthosis includes a heel end, a toe end, an arch section having a first end and a second end located between the heel end and the toe end and wherein the fifth layer extends from a point between the heel end and the first end of the arch section to a point adjacent the toe end.

11. The foot orthosis according to claim 10, wherein the fourth material is more rigid than the fifth.

12. The foot orthosis according to claim 1, wherein the vibrating device and the switch are located in the same layer.

13. The foot orthosis according to claim 1, wherein the vibrating device is located in a layer above the layer in which the switch is located.

14. The foot orthosis according to claim 1, wherein the vibrating device is located in a layer below the layer in which the switch is located.

15. The foot orthosis according to claim 1, wherein the controller is located between the fourth and fifth layers.

16. The foot orthosis according to claim 1, wherein the vibrating device is a vibrating motor.

17. The foot orthosis according to claim 1, wherein the orthosis is configured to fit within an article of footwear and is removable therefrom.

18. The foot orthosis according to claim 1, wherein the orthosis is integral with an article of footwear.

19. The foot orthosis according to claim 1, wherein the controller is located in one of the layers.

20. The foot orthosis according to claim 1, wherein the controller communicates with the vibrating device via a wireless connection.

21. A foot orthosis, including:
a power source;
a vibrating device;
a switch for activating the vibrating device; and
a programmable controller for controlling the frequency and duration of vibration of the vibrating device upon activation of the switch.

22. The foot orthosis according to claim 21, wherein the switch is a force-sensitive switch.

23. The foot orthosis according to claim 21, further including an article of footwear.

24. The foot orthosis according to claim 23, wherein the vibrating device and switch are integral with the article of footwear.

25. The foot orthosis according to claim 23, wherein the power source is integral with the article of footwear.

26. The foot orthosis according to claim 23, wherein the controller is integral with the article of footwear.

27. The foot orthosis according to claim 21, wherein the orthosis has an upper surface and a lower surface, the switch is located on the upper surface and the vibrating device is located on the lower surface.

28. A foot orthosis, including:
means for producing vibration;
means for activating the means for producing vibration;
means for controlling the frequency and duration of the vibration produced by the means for producing vibration; and
means for securing the means for vibration adjacent a human foot.
